SEQUENCE LISTING (15 sequences)

SEQ ID NO: 1 - protein, 99 aa, Artificial Sequence
  Description of Artificial Sequence: Synthetic consensus sequence
RRVGDELEKEYERAFSSFSAQLHVTPTTARELFGQVATQLFSDGNINWGRVVALFSFGGFLALKLVDKELEDLVSRLASFLSEFLAKTLANWLRENGGW

SEQ ID NO: 2 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 3 - protein, 25 aa, Homo sapiens
TGYDNREIVMKYIHYKLSQRGYEWD

SEQ ID NO: 4 - protein, 175 aa, Homo sapiens
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSGWMTFLEVTGKICEMLSLLKQYC

SEQ ID NO: 5 - protein, 30 aa, Homo sapiens
FGYIYRLAQDYLQCVLQIPQPGSGPSKTSR

SEQ ID NO: 6 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 7 - protein, 27 aa, Homo sapiens
MSQSNRELVVDFLSYKLSQKGYSWSQF

SEQ ID NO: 8 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETRLADWIHSSGGWAEFTALYGDGALEEARRLREGNWASVRTVLTGAVALGALVTVGAFFASK

SEQ ID NO: 9 - protein, 24 aa, Homo sapiens
SAPDTRALVADFVGYKLRQKGYVC

SEQ ID NO: 10 - protein, 350 aa, Homo sapiens [sequence omitted]

SEQ ID NO: 11 - protein, 20 aa, Homo sapiens
DLYRQSLEIISRYLREQATG

SEQ ID NO: 12 - protein, 24 aa, Artificial Sequence
  Description of Artificial Sequence: Synthetic consensus sequence
PRLDIRGLVVDYVTYKLSQNGYEW

SEQ ID NO: 13 - skipped

SEQ ID NO: 14 - DNA, 899 bp, Homo sapiens
agcctacgcacgaaagtgactaggaggaaggatattataaagtgatgcaaacagaaattccaccagcctccatgtatcatcatgtgtcataactcagtcaagctcagtgagcattctcagcacattgcctcaacagcttcaaggtgagccagctcaagactttgctctccaccaggcagaagatgacagactgtgaatttggatatatttacaggctggctcaggactatctgcagtgcgtcctacagataccacaacctggatcaggtccaagcaaaacgtccagagtgctacaaaatgttgcgttctcagtccaaaaagaagtggaaaagaatctgaagtcatgcttggacaatgttaatgttgtgtccgtagacactgccagaacactattcaaccaagtgatggaaaaggagtttgaagacggcatcattaactggggaagaattgtaaccatatttgcatttgaaggtattctcatcaagaaacttctacgacagcaaattgccccggatgtggatacctataaggagatttcatattttgttgcggagttcataatgaataacacaggagaatggataaggcaaaacggaggctgggaaaatggctttgtaaagaagtttgaacctaaatctggctggatgacttttctagaagttacaggaaagatctgtgaaatgctatctctcctgaagcaatactgttgaccagaaaggacactccatattgtgaaaccggcctaatttttctgactgatatggaaacgattgccaacacatacttctacttttaaataaacaactttgatgatgtaacttgaccttccagagttatggaaattttgtccccatgtaatgaataaattgtatgtatttttctctataaaaaaaaaaaaa

SEQ ID NO: 15 - protein, 175 aa, Homo sapiens; identical to SEQ ID NO: 4

(19) United States
(12) Patent Application Publication
(10) Pub. No.: US 2010/0297094 A1
(43) Pub. Date: Nov. 25, 2010

(54) THERAPY OR PREVENTION OF DISEASES WITH CELLS OR CELL SUPERNATANT

(76) Inventors: John M. Harlan, Seattle, WA (US); Robert K. Winn, Bainbridge Island, WA (US)

Correspondence Address: University of Washington/Fenwick, Silicon Valley Center, 801 California Street, Mountain View, CA, 94041, US

(21) Appl. No.: 12/697,982

(22) Filed: Feb. 1, 2010

Related U.S. Application Data
(60) Provisional application No. 61/148,754, filed on Jan. 30, 2009.

(51) Int. Cl. A61K 35/26; A61K 35/12; A61P 9/10; A61P 3/10; A61P 37/06; A61P 35/00; A61P 29/00; A61P 11/00

(52) U.S. Cl. 424/93.71; 424/93.1; 424/93.7

(57) ABSTRACT

Methods and compositions for the treatment or prevention of diseases or disorders including media conditioned by mesenchymal stem cells and BCL2A1 are provided. Methods for the production of therapeutic or preventative compositions contacted with BCL2A1 are also provided.

### CROSS REFERENCE TO RELATED APPLICATIONS

This application claims the benefit of U.S. Provisional Application No. 61/148,754 filed Jan. 30, 2009, which is incorporated by reference in its entirety.

This application is related to U.S. patent application Ser. No. 12/514,436 filed ______ and titled “Methods Of Inhibiting Cell Death Or Inflammation In A Mammal”, which is a U.S. 35 USC §371 of international Application No. PCT/US2007/084052, filed Nov. 8, 2007 which is a nonprovisional of U.S. Provisional Application No. 60/857,913, filed Nov. 10, 2006. This application is also related to U.S. application Ser. No. 11/576,591 filed Jan. 18, 2008 which is a U.S. 35 USC §371 of international Application No. PCT/US2005/035666, filed Oct. 4, 2005 which claims priority to U.S. Provisional Application No. 60/714,511 filed Oct. 4, 2004 and U.S. Provisional Application No. 60/709,053 filed Aug. 16, 2005. This application is also related to U.S. Provisional Application No. 61/148,768 filed Jan. 30, 2009, the contents of which are all hereby incorporated by reference.

### STATEMENT REGARDING FEDERALLY SPONSORED RESEARCH OR DEVELOPMENT

This invention was made with government support under Grant Nos. GM66197, GM42686 and HL72262 awarded by the National Institutes of Health. The government has certain rights in the invention.

### FIELD

The invention relates to methods and compositions for the treatment or prevention of diseases or disorders.

### BACKGROUND

Ischemia and reperfusion injury is present in a wide spectrum of clinically important disorders including traumatic injury, stroke, myocardial infarction, organ transplantation, mesenteric and peripheral vascular disease, and the like. Therapy directed at ischemia-reperfusion injury could have a significant impact on health in the United States and world-wide. For example, both stroke and myocardial infarction have a component of ischemia-reperfusion injury and together claim close to 800,000 lives annually. Likewise, it is estimated that 25,000 to 40,000 of the 160,000 death resulting from traumatic injuries could benefit from therapy of this type. These patients, particularly those receiving percutaneous interventions or thrombolytic therapy, might benefit from treatment designed to treat ischemia-reperfusion injury.

Accordingly, there is a need for the prevention and/or treatment of injury resulting from ischemia and/or reperfusion. The present invention addresses this and other needs.

### SUMMARY

Disclosed herein is a method of treating or preventing a disease or disorder, including administering a therapeutically or prophylactically effective amount of a composition to a subject that includes the disease or disorder, wherein the composition includes an isolated cell contacted with BCL2.

In one aspect, the disease or disorder can include an ischemia and reperfusion injury, ischemia, reperfusion injury, myocardial infarction, graft rejection, skin-graft rejection, cancer, melanoma, acute renal failure, multiple sclerosis, diabetes, rheumatoid arthritis (RA), retinal degeneration, acute lung injury, radiation trauma, insulin-dependent (Type 1) diabetes, or hepatic failure.

In one aspect, BCL2 is a recombinant BCL2, or BCL2 is a human BCL2, or BCL2 includes a BCL2 protein, or BCL2 includes a BCL2 nucleic acid.

In another aspect, BCL2 is BCL2A1. In another aspect, the BCL2 is selected from the group consisting of: (a) a protein comprising an amino acid sequence that is at least 35% identical to the amino acid sequence set forth in SEQ ID NO:1; (b) a protein comprising at least 12 amino acids, wherein the protein is at least 50% similar to a segment of a Bcl-2 protein, wherein the Bcl-2 protein consists of the amino acid sequence set forth in SEQ ID NO:2; (c) a protein comprising at least 12 amino acids, wherein the protein is at least 50% similar to a segment of an A-1 protein, wherein the A-1 protein consists of the amino acid sequence set forth in SEQ ID NO:4; (d) a protein comprising at least 12 amino acids, wherein the protein is at least 50% similar to a segment of a Bcl-X protein, wherein the Bcl-X protein consists of the amino acid sequence set forth in SEQ ID NO:6; (e) a protein comprising at least 12 amino acids, wherein the protein is at least 50% similar to a segment of a Bcl-W protein, wherein the Bcl-W protein consists of the amino acid sequence set forth in SEQ ID NO:8; (f) a protein comprising at least 12 amino acids, wherein the protein is at least 50% similar to a segment of an Mcl-1 protein, wherein the Mcl-1 protein consists of the amino acid sequence set forth in SEQ ID NO:10; and (g) a protein that is at least 50% similar to a BH4 domain consisting of the amino acid sequence set forth in SEQ ID NO:12.

In other aspects, BCL2A1 is a recombinant BCL2A1, or BCL2A1 is a human BCL2A1, or BCL2A1 includes a BCL2A1 protein, or BCL2A1 includes a BCL2A1 nucleic acid.

In another aspect, the subject is a human.

In other aspects, the cell can include a mesenchymal stem cell (MSC), a dendritic cell, a monocyte, a macrophage, a myeloid cell, a THP-1 cell, a JAWS-II cell, a leukocyte, a stem cell, or an immune cell.

In another aspect, the administration can be mucosal, parenteral, transdermal, subcutaneous, intravenous, bolus injection, intramuscular, or intraarterial administration to the subject.

Further disclosed herein is a method of treating or preventing a disease or disorder, including administering a therapeutically or prophylactically effective amount of a composition to a subject that includes the disease or disorder, wherein the composition includes a supernatant component, and wherein the supernatant component is derived from a cell culture contacted with BCL2.

In one aspect, the supernatant component is isolated using centrifugation. In another aspect, the supernatant component is concentrated using ultra filtration.

Further disclosed herein is a method of treating an ischemia and reperfusion injury including administering a therapeutically effective amount of a composition to a human, wherein the composition includes an isolated MSC contacted with a recombinant human BCL2A1 protein or a supernatant component, wherein the supernatant component is derived from a MSC cell culture contacted with recombinant human BCL2 protein.

Further disclosed herein is a composition for treating or preventing a disease or disorder that includes an isolated cell contacted with BCL2.

In one aspect, the composition can include a pharmaceutically acceptable carrier.

Further disclosed herein is a composition for treating or preventing a disease or disorder that includes a supernatant component, wherein the supernatant component is derived from a cell culture contacted with BCL2.

Further disclosed herein is a composition for treating an ischemia and reperfusion injury in a human, including an isolated MSC contacted with a recombinant human BCL2 protein or a supernatant component, wherein the supernatant component is derived from a MSC cell culture contacted with recombinant human BCL2 protein.

Further disclosed herein is a method for preparing a composition, including incubating an isolated cell in a cell culture including BCL2, wherein the BCL2 is in a sufficient amount for obtaining a therapeutically or prophylactically effective amount of the composition.

In one aspect, the preparation method can further include isolating the cell from the cell culture. In one aspect, the preparation method can further include washing the cell. In another aspect, the preparation method can further include culturing the cell in a cell culture that does not include serum. In another aspect, the preparation method can further include suspending the cell in a pharmaceutically acceptable carrier. In another aspect, the preparation method can further include isolating a supernatant component from the cell culture. In another aspect, the preparation method can further include suspending the supernatant component in a pharmaceutically acceptable carrier.

In another aspect, the incubation of the cell with the cell culture including BCL2 is for a 4 hour time period at 37° C. In some aspects, the cells can be frozen or lypholized.

### BRIEF DESCRIPTION OF THE DRAWINGS

These and other features, aspects, and advantages of the present invention will become better understood with regard to the following description, and accompanying drawings, where:

FIG. 1 shows mouse hind limb tissue viability as measured by MTT assay following treatment of the mice with Jaws-II cells (available from ATCC) previously incubated with either recombinant human (rh)BCL2A1 or rhBim. The cells treated with rhBCL2A1 provided protection from 90 minutes of ischemia followed by 24 hours of reperfusion. Differences were significant by Mann-Whitney test at p<0.05.

FIG. 2 shows mouse hind limb tissue viability as measured by MTT assay following treatment with supernatant from Jaws-II cells previously incubated with either rhBCL2A1 or rhBim. Supernatant from rhBCL2A1 treated Jaws-II cells provided protection from 90 minutes of ischemia followed by 24 hours of reperfusion. Differences were significant by t-test at p<0.05.

FIG. 3 shows mouse hind limb tissue viability as measured by MTT assay following treatment with supernatant from human THP-1 cells previously incubated with either rhBCL2A1 or rhBim. Supernatant from rhBCL2A1 treated THP-1 cells provided protection from 90 minutes of ischemia followed by 24 hours of reperfusion. Differences were significant by t-test at p<0.05.

FIG. 4 shows mouse hind limb tissue viability as measured by MTT assay following treatment with supernatant from bone marrow derived macrophages (BMDMs) previously incubated with either rhBCL2A1 or rhBim. Supernatant from rhBCL2A1 treated BMDM provided protection from 90 minutes of ischemia followed by 24 hours of reperfusion. Differences were significant by Mann-Whitney test at p<0.05.

FIG. 5 shows mouse hind limb tissue viability as measured by MTT assay following treatment with supernatant from mesenchymal stem cells (MSCs) previously incubated with either rhBCL2A1 or saline. Supernatant from rhBCL2A1 treated MSCs provided protection from 90 minutes of ischemia followed by 24 hours of reperfusion. Differences were significant among all groups by the Non-parametric test (Kruskal-Wallis) at p<0.0001. The difference between MSCs given rhBCL2A1 vs saline was significant at p<0.001 and the difference between MSCs given rhBCL2A1 vs MSCs given Bim was significant at p<0.01 by non-parametric test (Dunn's multiple comparisons).

### DETAILED DESCRIPTION

Terms used in the claims and specification are defined as set forth below unless otherwise specified.

The term “stem cell” refers to any cell that has the ability to self renew and to differentiate into a variety of cell types.

The term “culture” or “cell culture” refers to one or more cells within a defined boundary such that the cell(s) are allotted space and growth conditions typically compatible with cell growth or sustaining its viability. Likewise, the term “culture,” used as a verb, refers to the process of providing said space and growth conditions suitable for growth of a cell or sustaining its viability.

The term “conditioned media” or “supernatant” refers to media that has been exposed to cells grown in culture for a time sufficient to include at least one additional component in the media, produced by the cells, that was not present in the starting media.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term “Bcl protein” refers to a protein that inhibits cell death in a mammal when administered to the mammal, and/or inhibits inflammation in a mammal when administered to the mammal, and that is a member of at least one of the following groups of proteins, identified as Groups (I) through (VII) below.

Group (I): A protein that includes an amino acid sequence that is at least 35% identical (e.g., at least 40%, or at least 45%, or at least 50%, or at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical)) to the amino acid sequence set forth in SEQ ID NO:1:

(SEQ ID NO: 1)
RRVGDELEKEYERAFSSFSAQLHVTPTTARELFGQVATQLFSDGNINWGR
VVALFSFGGFLALKLVDKELEDLVSRLASFLSEFLAKTLANWLRENGGW.

The amino acid sequence set forth in SEQ ID NO:1 is a consensus sequence for the Bcl domain for members of the Bcl-2 family of proteins.

Group (II): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of the Bcl-2 protein consisting of the amino acid sequence set forth in SEQ ID NO:2 (GenBank accession number AAH27258). In some aspects, the protein is at least 50% similar to the following segment of Bcl-2 protein: TGYDNREIVMKYIHYKLSQRGYEWD (SEQ ID NO:3). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-2 protein consisting of the amino acid sequence set forth in SEQ ID NO:2. The Bcl-2 class of proteins are intracellular cytoplasmic proteins that inhibit cell death (see, e.g., J. M. Adams and S. Cory, Science 281:1322-1326, 1998); S. Cory, et al., Oncogene 22:8590-8607, 2003).

Group (III): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of an A-1 protein (also referred to as a Bfl-1 protein), wherein the A-1 protein consists of the amino acid sequence set forth in SEQ ID NO:4 (GenBank accession number AAC50438). In some aspects, the protein is at least 50% similar to the following segment of A-1 protein: FGYIYRLAQDYLQCVLQIPQPGSGPSKTSR (SEQ ID NO:5). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the A-1 protein consisting of the amino acid sequence set forth in SEQ ID NO:4. A-1 proteins are homologs of Bcl-2 and are intracellular cytoplasmic proteins that inhibit apoptosis (see, e.g., A. Karsan, et al., Blood 87(8):3089-3096, Apr. 15, 1996; S. S. Choi et al., Mammalian Genome 8:781-782, 1997).

Group (IV): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of a Bcl-X protein, wherein the Bcl-X protein consists of the amino acid sequence set forth in SEQ ID NO:6 (GenBank accession number Q07817). In some aspects, the protein is at least 50% similar to the following segment of Bcl-X protein: MSQSNRELVVDFLSYKLSQKGYSWSQF (SEQ ID NO:7). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-X protein consisting of the amino acid sequence set forth in SEQ ID NO:6. Bcl-X proteins are homologs of Bcl-2 and are intracellular cytoplasmic proteins that inhibit apoptosis (see, e.g., L. H. Boise, et al., Cell 74:597-608, 1993.

Group (V): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of a Bcl-W protein consisting of the amino acid sequence set forth in SEQ ID NO:8 (GenBank accession number AAB09055). In some aspects, the protein is at least 50% similar to the following segment of Bcl-W protein: SAPDTRALVADFVGYKLRQKGYVC (SEQ ID NO:9). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-W protein consisting of the amino acid sequence set forth in SEQ ID NO:8. Bcl-W proteins are homologs of Bcl-2, and are intracellular cytoplasmic proteins that inhibit apoptosis (see, e.g., L. Gibson, et al., Oncogene 13:665-675, 1996).

Group (VI): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of an Mcl-1 protein, wherein the Mcl-1 protein consists of the amino acid sequence set forth in SEQ ID NO:10 (GenBank accession number AAF64255). In some aspects, the protein is at least 50% similar to the following segment of Mcl-1 protein: DLYRQSLEIISRYLREQATG (SEQ ID NO:11). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Mcl-1 protein consisting of the amino acid sequence set forth in SEQ ID NO:10. Mcl-1 proteins are homologs of Bcl-2 and are intracellular cytoplasmic proteins that inhibit apoptosis.

Group (VII): A protein that is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a BH4 domain consisting of the following amino acid sequence: PRLDIRGLVVDYVTYKLSQNGYEW (SEQ ID NO:12). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to the BH4 domain consisting of the amino acid sequence set forth in SEQ ID NO:12. BH4 (Bcl-2 homology domain 4) is an N-terminal domain found in Bcl-2, Bcl-X, and Bcl-W proteins. The amino acid sequence set forth in SEQ ID NO:12 is a consensus sequence for the BH4 domain of the Bcl-2 family of proteins.

As used herein, the term “protein” includes proteins having at least 12 amino acids.

As used herein in connection with proteins useful in the practice of the present invention, the term “segment” refers to at least 12 contiguous amino acids, and can include the complete amino acid sequence of a protein.

As used herein, “Bcl protein”, “BCL2”, and “Bcl-2” are used interchangeably unless otherwise noted.

Representative examples of amino acid sequences of Bcl-2 proteins, useful in the practice of the present invention, are set forth in the protein database accessible through the Entrez search tool of the National Center for Biotechnology Information, U.S. National Library of Medicine, 8600 Rockville Pike, Bethesda, Md. 20894, under the following accession numbers (the amino acid sequences of each of the identified Bcl-2 proteins are incorporated herein by reference): AAN17784.1; AAB17352.1; AAC53460.1; AAK15454.1; AAC53458.1; AAB17354.1; AAA82174.1; AAF88137.1; AAC72232.1; CAC10003.1; AAC53459.1; AAA19257.1; CAA57886.1; AAB17353.1; AAB96881.1; CAA58557.1; AAA82173.1; AAC15799.1; AAA51039.1; AAK15455.1; AAK31308.1; AAK31307.1; CAA80657.1; AAF89532.1; AAK31306.1; BAB85856.2; AAP35872.1; AAH19307.1; BAB71819.1; CAA80061.1; AAF33212.1; AAP36940.1; CAA04597.1; AAB07677.1; AAR92491.1; AAA37281.1; AAH68988.1; AAC60701.2; AAK92201.1; CAB92245.1; AAA37282.1; BAC33767.1; AAP47159.1; AAA77686.1; BAA01978.1; BAC37060.1; AAA77687.1; AAA53662.1; CAA29778.1; AAH27258.1; AA026045.1; AAB53319.1; BAC24136.1; AAA35591.1; CAA78018.1; BAC81344.1; BAD05044.1; AAA51814.1; AAA51813.1; AAN03862.1; CAA57844.1; AAH40369.1; BAB28740.1; BAB62748.1; AAH44130.1; AAH71291.1; AAK81706.1; AAH74505.1; AA064470.1; BAD32203.1; CAA57845.1; AA064468.1; AAH74021.1; AAC64200.1; AAB86430.1; AAB09056.1; BAB29912.1; BAB23468.1; AAB09055.1; BAA19666.2; AAH73259.1; CAF93123.1; AA013177.2; CAF96873.1; AAL35559.1; AAP21091.1; AAB97953.1; AAG02475.1; AAK55419.1; AAH27536.1; AAB97956.1; AAH28762.1; AAB97954.1; AA089009.1; AAP35767.1; AAH16281.1; AAC50438.1; AAC50288.1; CAG46735.1; AAP36152.1; CAG02784.1; CAA70566.1; AAF89533.1; AA022992.1; AAA03620.1; CAG46760.1; BAC40796.1; CAA73684.1; BAC53619.1; AAH55592.1; AAH66960.1; AAC48806.1; AAF71267.1; AAH04431.1; AA074828.1; AAA93066.1; AAA74466.1; CAA58997.1; CAG33700.1; BAB85810.1; AAH14175.1; AAA03619.1; AAF98242.1; AAM74949.1; CAD10744.1; AAF71760.1; AAD13295.1; AAH78835.1; AAA75200.1; AAC60700.2; AAH53380.1; AAH18228.1; BAB28776.1; AAA03622.1; AAD31644.1; AAF36411.1; AAC26327.1; AAM34436.1; CAE54428.1; AAH03839.1; AAH21638.1; AAH05427.1; AAC31790.1; BAC77771.1; AAA74467.1; AAF64255.1; AAP36208.1; AAP35286.1; AAH71897.1; AAH17197.1; AAF74821.1; AAD13299.1; AAG00896.1; AAH78871.1; AAH30069.1; AAAC53582.1; AAB87418.1; BAC21258.1; AAF09129.1; AAH63201.1; AAK06406.1; AAR84081.1; AAP36565.1; AAP35936.1; AAH06203.1; AAD51719.1; AAD31645.1; and AAC50142.1.

Bcl proteins include, for example, naturally-occurring Bcl proteins, synthetic Bcl proteins that can incorporate non-natural amino acids, and Bcl fusion proteins in which a protein, peptide, amino acid sequence, or other chemical structure, is attached to a portion (e.g., N-terminal or C-terminal) of a Bcl protein (described in detail below). Representative examples of proteins or chemical structures that can be fused to a Bcl protein include: human serum albumin, an immunoglobulin, polyethylene glycol, or other protein or chemical structure that, for example, increases the serum half-life of the Bcl protein, or increases the efficacy of the Bcl protein, or reduces the immunogenicity of the Bcl protein.

The term “sufficient amount” means an amount sufficient to produce a desired effect, e.g., an amount sufficient to modulate protein aggregation in a cell.

The term “treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a disease, condition or disorder as described herein. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a disease or disorder associated with a disease, condition or disorder as described herein, but does not yet experience or exhibit symptoms, inhibiting the symptoms of a disease or disorder (slowing or arresting its development), providing relief from the symptoms or side effects of a disease (including palliative treatment), and relieving the symptoms of a disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

The term “pharmaceutically acceptable” refers to molecular entities and compositions that do not produce adverse, allergic, or other untoward reactions when administered to an animal or a human. Supplementary active ingredients also can be incorporated into the compositions.

“Concomitant administration” of a known therapeutic agent (small molecule, drug, compound, cells, cell line, and the like) with the composition of the present invention means administration of the therapeutic agent (small molecule, drug, compound, cells, cell line, and the like) together with the composition at such time that both the known therapeutic agent (small molecule, drug, compound, cells, cell line, and the like) will have a therapeutic effect or diagnostic effect. Such concomitant administration can involve concurrent (i.e., at the same time), prior, or subsequent administration of the therapeutic agent (small molecule, drug, compound, cells, cell line, and the like) with respect to the administration of a composition of the present invention. BCL2 (for example, BCL2A1) generated cell supernatant/conditioned media administered to the subject, cells treated with BCL2 (for example BCL2A1) then administered to the subject or BCL2 (for example BCL2A1) given concomitant with cells are all contemplated treatment regimens. A person of ordinary skill in the art would have no difficulty determining the appropriate timing, sequence, and dosages of administration for particular drugs (or other compounds) together with compositions of the present invention.

The term “therapeutically effective time” refers to the period of time during which a therapeutically effective amount of a conditioned media or cells is administered, and that is sufficient to reduce one or more symptoms of a condition.

The term “condition” is used to refer to a disease and/or a response to injury (e.g., trauma, and the like) or treatment (e.g., surgery, transplantation of tissue from a donor, and the like).

The term “ameliorating” refers to any therapeutically beneficial result in the treatment of a disease state, e.g., an ischemic disease state, including prophylaxis, lessening in the severity or progression, remission, or cure thereof.

The term “therapeutically effective amount” is an amount that is effective to ameliorate a symptom of a disease. A therapeutically effective amount can be a “prophylactically effective amount” as prophylaxis can be considered therapy.

As used herein, the term “disease” has the meaning generally known and understood in the art and comprises any abnormal condition in the function or well being of a host individual. A diagnosis of a particular disease by a healthcare professional can be made by direct examination and/or consideration of results of one or more diagnostic tests.

The term “inflammatory” when used in reference to a disease, disorder or condition refers to a pathological process caused by, resulting from, or resulting in inflammation that is inappropriate and/or does not resolve in the normal manner. Inflammation results in response to an injury or abnormal stimulation caused by a physical, chemical, or biologic agent; these reactions include the local reactions and resulting morphologic changes, destruction or removal of the injurious material, and responses that lead to repair and healing. Inflammatory disease and conditions can be systemic or localized to particular tissues or organs.

It must be noted that, as used in the specification and the appended claims, the singular forms “a,” “an” and “the” include plural referents unless the context clearly dictates otherwise.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but can include other elements not expressly listed or inherent to such process or method. Further, unless expressly stated to the contrary, “or” refers to an inclusive or and not to an exclusive or. For example, a condition A or B is satisfied by any one of the following:A is true (or present) and B is false (or not present), A is false (or not present) and B is true (or present), and both A and B are true (or present).

It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a composition” includes a combination of two or more vaccines compositions, and the like.

“About” as used herein when referring to a measurable value such as an amount, a temporal duration, and the like, is meant to encompass variations of ±20% or ±10%, more preferably +5%, even more preferably ±1%, and still more preferably +0.1% from the specified value, as such variations are appropriate to perform the disclosed methods.

Exemplary Compounds

BCL2A1

BCL2A1 is typically used for contacting cells in the present invention. Typically BCL2A1 can refer to BCL2A1 protein or BCL2A1 nucleic acid. In one aspect, BCL2A1 is a human BCL2A1. In one aspect of the present invention, BCL2A1 protein can refer to naturally occurring BCL2A1 protein. In another aspect, BCL2A1 protein can refer to synthetic BCL2A1. In yet another aspect, BCL2A1 protein can refer to recombinant BCL2A1. In general, BCL2A1 proteins can be obtained by known recombinant or synthetic methods, such as described in U.S. Pat. Nos. 4,086,196 and 5,556,940, each incorporated herein by reference for all purposes, and others as described in more detail below.

Furthermore, BCL2A1 proteins can include allelic variants, species variants, and conservative amino acid substitution variants of BCL2A1 or BCL2A1 fragments. BCL2A1 proteins can also include full-length BCL2A1 as well as BCL2A1 fragments. Fragments of BCL2A1 protein variants, in amounts giving equivalent or similar biological activity to full length BCL2A1, can be used in the methods of the invention, if desired by one of ordinary skill in the art. Fragments of BCL2A1 can typically incorporate at least the amino acid residues of BCL2A1 necessary for a biological activity similar to that of full length BCL2A1.

In other aspects, alternative forms of BCL2A1 protein variants can incorporate from 1 to 5 or more amino acid substitutions that can improve BCL2A1 protein stability and half-life, such as the replacement of methionine residues with leucine or other hydrophobic amino acids that improve BCL2A1 protein stability against oxidation, in addition the replacement of some amino acids with trypsin-insensitive amino acids such as histidine or other amino acids that improves BCL2A1 protein stability against proteases are also contemplated.

In other aspects, BCL2A1 proteins can include fragments, variants, and functional analogs of BCL2A1 having a 60%, 70%, 80%, 90%, 95%, 96%, 97%, 98%, 99%, or more homologous amino acid sequence with BCL2A1 and fragments thereof. The present invention includes pharmaceutical formulations (see below) comprising, e.g., cells contacted with such BCL2A1 protein variants and functional analogs, carrying modifications like substitutions, deletions, insertions, inversions or cyclisations, but nevertheless having substantially similar biological activities of a full-length BCL2A1 protein.

In other aspects, BCL2A1 proteins useful in the methods of the present invention can include the use of a BCL2A1 protein such as:(a) full-length protein; (b) biologically active variants of full-length protein; (c) biologically active protein fragments; (d) biologically active variants of protein fragments; (e) biologically active variants having at least 75% homology with BCL2A1; (f) biologically active variants having at least 60% identity with BCL2A1; and (g) biologically active variants encoded by a nucleic acid sequence that hybridizes under stringent conditions to a complementary nucleic acid sequence of BCL2A1 or BCL2A1 fragments.

Production of BCL2A1 Proteins and Fragments Thereof.

BCL2A1 proteins can be generated wholly or partly by chemical synthesis (as described in detail below). The proteins of the invention can be readily prepared according to well-established, standard liquid or solid-phase protein synthesis methods, general descriptions of which are broadly available (see, for example, in J. M. Stewart and J. D. Young, Solid Phase Protein Synthesis, 2nd edition, Pierce Chemical Company, Rockford, Ill., 1984, in M. Bodanzsky and A. Bodanzsky, The Practice of Protein Synthesis, Springer Verlag, N.Y., 1984; and Applied Biosystems 430A Users Manual, ABI Inc., Foster City, Calif.), or they can be prepared in solution, by the liquid phase method or by any combination of solid-phase, liquid phase and solution chemistry, e.g., by first completing the respective protein portion and then, if desired and appropriate, after removal of any protecting groups being present, by introduction of a residue by reaction of the respective carbonic or sulfonic acid or a reactive derivative thereof.

The BCL2A1 proteins can also be obtained by methods well-known in the art for protein purification and recombinant protein expression. For recombinant expression of one or more of the proteins, the nucleic acid containing all or a portion of the nucleotide sequence encoding the BCL2A1 protein can be inserted into an appropriate expression vector (i.e., a vector that contains the necessary elements for the transcription and translation of the inserted protein coding sequence). In one aspect, the regulatory elements are heterologous (i.e., not the native gene promoter). Alternately, the necessary transcriptional and translational signals can also be supplied by the native promoter for the genes and/or their flanking regions.

The BCL2A1 proteins can also be purified from a natural source. Depending on the source, the BCL2A1 protein can be brought into a solution by breaking the tissue or cells containing it. There are several methods to achieve this, including:repeated freezing and thawing, sonication, homogenization by high pressure or permeabilization by organic solvents. The method of choice typically depends on how fragile the BCL2A1 protein is and how sturdy the cells are. After this extraction process soluble protein will be in the solvent, and can be separated from cell membranes, DNA, and the like by centrifugation. After the extraction process the protein of interest can be further purified using methods known in the art including precipitation, differential solubilization, ultracentrifugation, and/or chromatography methods including size exclusion, ion exchange, high pressure liquid, and immunoaffinity.

Cells

BCL2A1 contacted cells of the present invention (or cells contacted with any other BCL2 family member as described herein) can include any cell known in the art, e.g., eukaryotic cells, prokaryotic cells, mammalian cells, non-mammalian cells, embryonic cells, and non-embryonic cells. Other examples of cells can include:a MSC, a dendritic cell, a monocyte, a macrophage, a myeloid cell, a THP-1 cell, a JAWS-II cell, a leukocyte, a stem cell, or an immune cell. Cells of the present invention can typically be isolated cells.

Cells of the present invention can be obtained from any tissue, e.g., bone marrow, peripheral blood, skin, hair root, muscle or fat tissue, uterine endometrium, blood, umbilical cord tissue or blood and primary cultures of various tissues.

Mesenchymal Stem Cells (MSCs).

Cells of the present invention can include MSCs. In one aspect, an MSC can be isolated from bone marrow, although any source can be used for obtaining MSC for the present invention. By way of example, the bone marrow aspirate can be isolated, washed, and resuspended in media and placed into sterile culture in vitro. Initially, the isolated cells can be plated with serum in the media. The MSC typically adhere to the culture dish while essentially all other cells are nonadherent and are removed by rinsing (Friedenstein, Exp. Hematol. 4:267-74, 1976). MSC will typically grow and expand in culture, yielding a well-defined population of pluripotent stem cells. MSC can be further depleted of CD45 positive (+) cells, by example FACS, to remove residual macrophages or other hematopoietic cell lineages prior to further expansion, production of MSC conditioned media (CM), or MSC administration to a subject. In one aspect, the MSC of the present invention can be CD34, CD45 negative; in another aspect the MSC are SH2, SH4, CD29, CD44, CD71, CD90, CD106, CD120a positive, and CD124, CD14, CD34, CD45 negative. In addition to adherence separation, MSC can be isolated by any technique known to one of skill in the art, including but not limited to, density gradient fractionation, immunoselection, leukapheresis, and the like.

The MSC can also be tested morphologically and functionally to show that the isolated stem cells are MSC or similar cells. For example, a portion of the cells can be cultured in differentiation media to differentiate the MSC into osteocytes and adipocytes as described by Pittenger et al., Science 284:143-147, 1999. The remaining MSC can be further expanded in culture for administration to a subject, for generation of CM, or for cryopreservation for later use.

Other cells derived from the MSC described herein by differentiation in vitro can also be used in the present invention. The other cells can be used for delivery to the subject as described for the MSC or in combination with MSC or CM, and combinations thereof.

Cells of the present invention can be derived from the subject to be administered the cell or, under defined circumstances, from a compatible but allogeneic donor subject. Donor stem cells can be used from a donor having similar compatibility as defined for the subject to be transplanted, including, e.g., HLA compatibility, known to one skilled in the art. Since cells can be expanded in vitro, multiple administrations of cells are possible to further augment the therapeutic effect of the cell. Use of autologous cells can eliminate concerns regarding immune tolerance, among other things.

The cells of the present invention can be genetically modified prior to administration to the patient or prior to generation of CM, discussed below. The cells can be genetically modified using addition (e.g., BCL2A1 or other genes with similar function to BCL2A1 as are known in the art) or deletion of genes; whose products are known to support or inhibit cellular survival, apoptosis, stimulate cell migration and proliferation, to exert anti-inflammatory actions, and/or to improve hemodynamics. Expression of the genes delivered to the cell can be placed under the control of various promoters, including, but not limited to drug-sensitive promoters that allow both controlled activation and inactivation of genes. Cloning of the expression vectors for genetically modifying the cells is typically performed using materials and methods known to one of skill in the art. Genetic modification of the cells can be accomplished using methods known to one of skill in the art, including lipofection, calcium phosphate precipitation, infection, including viral vectors, electroporation, and the like. Other methods of genetic expression or inhibition of target genes are generally well known to one of skill in the art and can include use of, e.g., small interfering RNA, micro RNA, antisense RNA or DNA, and the like.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, Sambrook, et al., 1989, Molecular Cloning:A Laboratory Manual, Second Edition, Books 1-3, Cold Spring Harbor Laboratory Press; Ausubel, F. M. et al. (1995 and periodic supplements); Current Protocols in Molecular Biology, ch. 9, 13, and 16, John Wiley & Sons, New York, N.Y.); B. Roe, J. Crabtree, and A. Kahn, 1996, DNA Isolation and Sequencing:Essential Techniques, John Wiley & Sons; J. M. Polak and James 0′ D. McGee, 1990, Oligonucleotide Synthesis:A Practical Approach, IRL Press; D. M. J. Lilley and J. E. DaWberg, 1992, Methods of Enzymology: DNA Structure Part A: Synthesis and Physical Analysis of DNA Methods in Enzymology, Academic Press; Using Antibodies:A Laboratory Manual:Portable Protocol NO. I by Edward Harlow, David Lane, Ed Harlow (1999, Cold Spring Harbor Laboratory Press, I SBN 0-87969-544-7); Antibodies:A Laboratory Manual by Ed Harlow (Editor), David Lane (Editor) (1988, Cold Spring Harbor Laboratory Press, I SBN 0-87969-314-2), 1855; and Lab Ref:A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts (as well as the current editions) is herein incorporated by reference.

Cell Conditioned Media

Cell Conditioned Media (CM) can be obtained by culturing an effective amount of BCL2A1 and the cell described above for a time sufficient to condition the media. In one aspect of the present invention, the CM is MSC CM. By way of non-limiting example, the CM can be obtained as follows: cells can be obtained as described above and the cells plated in culture. As an example, MSCs that have been depleted of other cells types, for example by adherence plating and removal of CD45 positive cells by FACS sorting, can be grown to substantially confluent cultures that are essentially contact inhibited. As the cultures of cells are expanding, the cells can be grown in media containing serum. The cells can also be grown with autologous serum from the cell or CM recipient subject. Once the cultures have expanded to high subconfluence, i.e., about 3−5×10⁶ cells/T-75 flask or as otherwise desirable to one of skill in the art, serum can be removed from the media if serum free CM is desired by one of skill. Typically an effective amount of BCL2A1 can be added to the culture at any time during the culture period, e.g., prior to cell confluence or after cell confluence is reached. After addition of BCL2A1 to the cells of the present invention the media will typically contain one or more components with therapeutic efficacy, such as an effective supernatant component. Generally the identity of an effective supernatant component is easily identifiable to one of skill in the art using methods and techniques that are typically available to one of skill, such as mass spectrometry, HPLC, GC/MS, electrophoresis, recombinant cloning techniques, and the like.

The cells can be grown in normal oxygen conditions, i.e., room air+5% CO₂ (also, e.g., pO₂ approximately 21%). Alternatively, the cells can be grown under hypoxic conditions (e.g., pO₂ 5%). The media can be incubated in the presence of the cells for a time sufficient to add at least one component to the media that was not present prior to addition of the media to the culture, e.g., a BCL2A1 protein or a BCL2A1 nucleic acid or a similar protein or gene. In one aspect, the media can be conditioned for one or more minutes. In some such aspects, the media can be conditioned for 5, 10, 15, 20, 30, 60, 120, 300, or more minutes. In another aspect, the media can be conditioned for days or weeks. In some such aspects, the media can be conditioned for one, two, three, four, five, six, seven or more days.

In another aspect the media can be conditioned for one to three days or more, in another aspect the media can be conditioned for two days, and in another aspect the media can be conditioned for one week. The CM can be collected and filtered though a small pore filter, such as e.g., a 0.22 μM filter, to sterilize the CM and to remove any particulates that are unwanted or extraneous. The CM can also be centrifuged to remove unwanted or excess components. The CM can be administered to the subject or the CM can be frozen, for example at −120° C., and stored for later administration. The CM can also be concentrated, for example by centrifugation, dialysis, filtration, lyophilization, and the like.

Presence of at least one component added to the media by the cells can be confirmed in vitro using a biological assay, ELISA, or a separation analysis, such as HPLC. The CM can also be tested in vivo. As described below, CM can be administered in single, multiple, or continuous administrations or combinations thereof. The generation and use of the cells and CM is described in more detail in the examples provided below.

Peptides

The invention provides composition for treating or preventing a disease or disorder, comprising an isolated cell contacted with a BCL2 peptide. Exemplary peptides of the invention have an amino acid sequence including those listed herein, and analogs, derivatives, amidated variations and conservative variations thereof, wherein the peptides have activity. The peptides of the invention include the sequences provided herein, as well as the broader groups of peptides having hydrophilic and hydrophobic substitutions, and conservative variations thereof.

“Isolated” when used in reference to a peptide, refers to a peptide substantially free of proteins, lipids, nucleic acids, for example, with which it can be naturally associated. Those of skill in the art can make similar substitutions to achieve peptides with greater biological activity. For example, the invention includes the peptides depicted in sequences provided herein, as well as analogs or derivatives thereof, as long as the bioactivity (e.g., treating or preventing a disease or disorder) of the peptide remains. Minor modifications of the primary amino acid sequence of the peptides of the invention can result in peptides that have substantially equivalent activity as compared to the specific peptides described herein. Such modifications can be deliberate, as by site-directed mutagenesis, or can be spontaneous. All of the peptides produced by these modifications are included herein as long as the biological activity of the original peptide still exists.

Further, deletion of one or more amino acids can also result in a modification of the structure of the resultant molecule without significantly altering its biological activity. This can lead to the development of a smaller active molecule that would also have utility. For example, amino or carboxy terminal amino acids that can not be required for biological activity of the particular peptide can be removed. Peptides of the invention include any analog, homolog, mutant, isomer or derivative of the peptides disclosed in the present invention, so long as the bioactivity as described herein remains. All peptides were synthesized using L amino acids, however, all D forms of the peptides can be synthetically produced. In addition, C-terminal derivatives can be produced, such as C-terminal methyl esters and C-terminal amidates, in order to increase the biological activity of a peptide of the invention. The peptide can be synthesized such that the sequence is reversed whereby the last amino acid in the sequence becomes the first amino acid, and the penultimate amino acid becomes the second amino acid, and so on. It is well known that such reversed peptides usually have similar biological activities to the original sequence.

In certain aspects, the peptides of the invention include peptide analogs and peptide mimetics. Indeed, the peptides of the invention include peptides having any of a variety of different modifications, including those described herein.

Peptide analogs of the invention are generally designed and produced by chemical modifications of a lead peptide, including, e.g., any of the particular peptides described herein, such as any of the following sequences disclosed. The present invention further encompasses polypeptides up to about 50 amino acids in length that include the amino acid sequences and functional variants or peptide mimetics of the sequences described herein.

In another aspect, a peptide of the present invention is a pseudopeptide. Pseudopeptides or amide bond surrogates refers to peptides containing chemical modifications of some (or all) of the peptide bonds. The introduction of amide bond surrogates not only decreases peptide degradation but also can significantly modify some of the biochemical properties of the peptides, particularly the conformational flexibility and hydrophobicity.

To improve or alter the characteristics of polypeptides of the present invention, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the polypeptides of the present invention can be produced as multimers including dimers, trimers and tetramers. Multimerization can be facilitated by linkers, introduction of cysteines to permit creation of interchain disulphide bonds, or recombinantly though heterologous polypeptides such as Fc regions.

It is known in the art that one or more amino acids can be deleted from the N-terminus or C-terminus without substantial loss of biological function. See, e.g., Ron et al., Biol. Chem. 268:2984-2988, 1993. Accordingly, the present invention provides polypeptides having one or more residues deleted from the amino terminus. Similarly, many examples of biologically functional C-terminal deletion mutants are known (see, e.g., Dobeli et al., J. Biotechnology 7:199-216, 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides which show substantial chaperone polypeptide activity. Such mutants include deletions, insertions, inversions, repeats, and substitutions selected according to general rules known in the art so as to have little effect on activity.

There are two main approaches for studying the tolerance of an amino acid sequence to change, see, Bowie et al., Science 247:1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the polypeptide of the present invention can be, for example:(i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the polypeptides of the present invention can include one or more amino acid substitutions, deletions, or additions, either from natural mutations or human manipulation. As indicated, changes are preferably of a minor nature, such as conservative amino acid substitutions that do not significantly affect the folding or activity of the protein.

The following groups of amino acids represent equivalent changes:(1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Furthermore, polypeptides of the present invention can include one or more amino acid substitutions that mimic modified amino acids. An example of this type of substitution includes replacing amino acids that are capable of being phosphorylated (e.g., serine, threonine, or tyrosine) with a negatively charged amino acid that resembles the negative charge of the phosphorylated amino acid (e.g., aspartic acid or glutamic acid). Also included is substitution of amino acids that are capable of being modified by hydrophobic groups (e.g., arginine) with amino acids carrying bulky hydrophobic side chains, such as tryptophan or phenylalanine. Therefore, a specific aspect of the invention includes polypeptides that include one or more amino acid substitutions that mimic modified amino acids at positions where amino acids that are capable of being modified are normally positioned. Further included are polypeptides where any subset of modifiable amino acids is substituted. For example, a polypeptide that includes three serine residues can be substituted at any one, any two, or all three of said serines. Furthermore, any polypeptide amino acid capable of being modified can be excluded from substitution with a modification-mimicking amino acid.

The present invention is further directed to fragments of the polypeptides of the present invention.

In addition, it should be understood that in certain aspects, the peptides of the present invention include two or more modifications, including, but not limited to those described herein. By taking into the account the features of the peptide drugs on the market or under current development, it is clear that most of the peptides successfully stabilized against proteolysis consist of a mixture of several types of the above described modifications. This conclusion is understood in the light of the knowledge that many different enzymes are implicated in peptide degradation.

Peptides, Peptide Variants, and Peptide Mimetics

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer Amino acid mimetics refers to chemical compounds that have a structure that is different from the general chemical structure of an amino acid, but which functions in a manner similar to a naturally occurring amino acid. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

“Peptide” as used herein includes peptides that are conservative variations of those peptides specifically exemplified herein. “Conservative variation” as used herein denotes the replacement of an amino acid residue by another, biologically similar residue. Examples of conservative variations include, but are not limited to, the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acids, or glutamine for asparagine, and the like. Neutral hydrophilic amino acids that can be substituted for one another include asparagine, glutamine, serine and threonine. The term “conservative variation” also includes the use of a substituted amino acid in place of an unsubstituted parent amino acid provided that antibodies raised to the substituted polypeptide also immunoreact with the unsubstituted polypeptide. Such conservative substitutions are within the definition of the classes of the peptides of the invention. “Cationic” as is used to refer to any peptide that possesses sufficient positively charged amino acids to have a pI (isoelectric point) greater than about 9.0.

The biological activity of the peptides can be determined by standard methods known to those of skill in the art, such as “minimal inhibitory concentration (MIC)” assay described in the present examples, whereby the lowest concentration at which no change in OD is observed for a given period of time is recorded as MIC.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, e.g., a polypeptide fragment of an Bcl protein having Bcl activity as described herein.

Polypeptide mimetic compositions can contain any combination of non-natural structural components, which are typically from three structural groups:a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH2- for —C(═O)—NH—), aminomethylene (CH2-NH), ethylene, olefin (CH═CH), ether (CH2-O), thioether (CH2-S), tetrazole (CN4-), thiazole, retroamide, thioamide, or ester (see, e.g., Spatola, 1983, Chemistry and Biochemistry of Amino Acids, Peptides and Proteins 7:267-357).

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, guanidino-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a Bcl polypeptide having biological activity of the invention, resulting in modification of the structure of the polypeptide, without significantly altering its biological activity. For example, amino- or carboxyl-terminal, or internal, amino acids that are not required for biological activity can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9:205-223, 1998; Hruby, Curr. Opin. Chem. Biol. 1:114-119, 1997; Ostergaard, Mol. Divers. 3:17-27, 1997; Ostresh, Methods Enzymol. 267:220-234, 1996. Modified peptides of the invention can be further produced by chemical modification methods, see, e.g., Belousov, Nucleic Acids Res. 25:3440-3444, 1997; Frenkel, Free Radic. Biol. Med. 19:373-380, 1995; Blommers, Biochemistry 33:7886-7896, 1994.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, 1995, Therapeutic Peptides and Proteins, Formulation, Processing and Delivery Systems. For example, peptide synthesis can be performed using various solid-phase techniques (see e.g., Roberge, Science 269:202, 1995; Merrifield, Methods Enzymol. 289:3-13, 1997) and automated synthesis can be achieved, e.g., using the ABI 431A Peptide Synthesizer (Perkin Elmer) in accordance with the instructions provided by the manufacturer.

Peptides of the invention can be synthesized by such commonly used methods as t-BOC or FMOC protection of alpha-amino groups. Both methods involve stepwise syntheses whereby a single amino acid is added at each step starting from the C terminus of the peptide (See, Coligan et al., Current Protocols in Immunology, Wiley Interscience, 1991, Unit 9). Peptides of the invention can also be synthesized by the well known solid phase peptide synthesis methods described in Merrifield, J. Am. Chem. Soc. 85:2149, 1962, and Stewart and Young, 1969, Solid Phase Peptides Synthesis 27-62, using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with 1% acetic acid solution which is then lyophilized to yield the crude material. This can normally be purified by such techniques as gel filtration on Sephadex G-15 using 5% acetic acid as a solvent. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation.

Analogs, polypeptide fragment of Bcl protein having biological activity as described herein, are generally designed and produced by chemical modifications of a lead peptide, including, e.g., any of the particular peptides described herein, such as any of the sequences described herein.

The terms “identical” or percent “identity”, in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., 1995 supplement, Current Protocols in Molecular Biology).

Programs for searching for alignments are well known in the art, e.g., BLAST and the like. For example, if the target species is human, a source of such amino acid sequences or gene sequences (germline or rearranged antibody sequences) can be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain aspects, an acceptable range of homology is greater than about 50%. It should be understood that target species can be other than human.

A preferred example of algorithm that is suitable for determining percent sequence identity and sequence similarity are the BLAST and BLAST 2.0 algorithms, which are described in Altschul et al., Nuc. Acids Res. 25:3389-3402, 1977 and Altschul et al., J. Mol. Biol. 215:403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when:the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915, 1989) alignments (B) of 50, expectation (E) of 10, M=5, N=−4, and a comparison of both strands.

Polypeptides and Functional Variants Thereof

“Polypeptide” includes proteins, fusion proteins, oligopeptides and polypeptide derivatives, with the exception that peptidomimetics are considered to be small molecules herein.

A “protein” is a molecule having a sequence of amino acids that are linked to each other in a linear molecule by peptide bonds. The term protein refers to a polypeptide that is isolated from a natural source, or produced from an isolated cDNA using recombinant DNA technology; and has a sequence of amino acids having a length of at least about 200 amino acids.

A “fusion protein” is a type of recombinant protein that has an amino acid sequence that results from the linkage of the amino acid sequences of two or more normally separate polypeptides.

A “protein fragment” is a proteolytic fragment of a larger polypeptide, which can be a protein or a fusion protein. A proteolytic fragment can be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” or “peptide” is a polypeptide having a short amino acid sequence (i.e., 2 to about 200 amino acids). An oligopeptide is generally prepared by chemical synthesis.

Although oligopeptides and protein fragments can be otherwise prepared, it is possible to use recombinant DNA technology and/or in vitro biochemical manipulations. For example, a nucleic acid encoding an amino acid sequence can be prepared and used as a template for in vitro transcription/translation reactions. In such reactions, an exogenous nucleic acid encoding a preselected polypeptide is introduced into a mixture that is essentially depleted of exogenous nucleic acids that contains all of the cellular components required for transcription and translation. One or more radiolabeled amino acids are added before or with the exogenous DNA, and transcription and translation are allowed to proceed. Because the only nucleic acid present in the reaction mix is the exogenous nucleic acid added to the reaction, only polypeptides encoded thereby are produced, and incorporate the radiolabeled amino acid(s). In this manner, polypeptides encoded by a preselected exogenous nucleic acid are radiolabeled. Although other proteins are present in the reaction mix, the preselected polypeptide is the only one that is produced in the presence of the radiolabeled amino acids and is thus uniquely labeled.

As is explained in detail below, “polypeptide derivatives” include without limitation mutant polypeptides, chemically modified polypeptides, and peptidomimetics.

The polypeptides of this invention, including the analogs and other modified variants, can generally be prepared following known techniques. Preferably, synthetic production of the polypeptide of the invention can be according to the solid phase synthetic method. For example, the solid phase synthesis is well understood and is a common method for preparation of polypeptides, as are a variety of modifications of that technique. Merrifield, J. Am. Chem. Soc., 85:2149, 1964; Stewart and Young, 1984, Solid Phase polypeptide Synthesis; Bodansky and Bodanszky, 1984, The Practice of polypeptide Synthesis; Atherton and Sheppard, 1989, Solid Phase polypeptide Synthesis:A Practical Approach.

Alternatively, polypeptides of this invention can be prepared in recombinant systems using polynucleotide sequences encoding the polypeptides.

A “variant” or “functional variant” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention can contain more than one of the following modifications within the same polypeptide. Preferred polypeptide derivatives retain a desirable attribute, which can be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

A polypeptide having an amino acid sequence identical to that found in a protein prepared from a natural source is a “wild type” polypeptide. Functional variants of polypeptides can be prepared by chemical synthesis, including without limitation combinatorial synthesis.

Functional variants of polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences can comprise substitutions, deletions and additions of amino acids, with the proviso that such amino acids are naturally occurring amino acids.

Thus, subjecting a nucleic acid that encodes a polypeptide to mutagenesis is one technique that can be used to prepare Functional variants of polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis can be randomly targeted (i.e., mutations can occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids can be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences can be prepared by directed mutagenesis.

As contemplated by this invention, “polypeptide” includes those having one or more chemical modification relative to another polypeptide, i.e., chemically modified polypeptides. The polypeptide from which a chemically modified polypeptide is derived can be a wild type protein, a functional variant protein or a functional variant polypeptide, or polypeptide fragments thereof; an antibody or other polypeptide ligand according to the invention including without limitation single-chain antibodies, crystalline proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum. Powell et al., Pharma. Res. 10:1268-1273, 1993. Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. 1993, cited above).

Different host cells will contain different post-translational modification mechanisms that can provide particular types of post-translational modification of a fusion protein if the amino acid sequences required for such modifications is present in the fusion protein. A large number (about 100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and can influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation can vary depending on which host cells are used to produce the fusion protein.

Another type of post-translation modification is the phosphorylation of a free hydroxyl group of the side chain of one or more Ser, Thr or Tyr residues, Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which can have a different chemical version of the methionine residue encoded by a start codon, and these will result in amino termini with different chemical modifications.

For example, many or most bacterial proteins are synthesized with an amino terminal amino acid that is a modified form of methionine, i.e., N-formyl-methionine (fMet). Although the statement is often made that all bacterial proteins are synthesized with an fMet initiator amino acid; although this can be true for E. coli, recent studies have shown that it is not true in the case of other bacteria such as Pseudomonas aeruginosa. Newton et al., J. Biol. Chem. 274:22143-22146, 1999. In any event, in E. coli, the formyl group of fMet is usually enzymatically removed after translation to yield an amino terminal methionine residue, although the entire fMet residue is sometimes removed (see Hershey, 1987, Escherichia coli and Salmonella Typhimurium: Cellular and Molecular Biology, 1:613-647, and references cited therein.). E. coli mutants that lack the enzymes (such as, e.g., formylase) that catalyze such post-translational modifications will produce proteins having an amino terminal fMet residue (Guillon et al., J. Bacteria 174:4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23:263-267, 1998; and Driessen et al., CRC Crit. Rev. Biochem. 18:281-325, 1985) Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al., Annu. Rev. Physiol. 50:333-344, 1988, and Bradbury et al., Lung Cancer 14:239-251, 1996. About 50% of known endocrine and neuroendocrine peptide hormones are alpha-amidated (Treston et al., Cell Growth Differ. 4:911-920, 1993). In most cases, carboxy alpha-amidation is required to activate these peptide hormones.

Polypeptide Mimetic

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself. For example, polypeptides can exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean, BioEssays 16:683-687, 1994; Cohen and Shatzmiller, J. Mol. Graph. 11:166-173, 1993; Wiley and Rich, Med. Res. Rev. 13:327-384, 1993; Moore, Trends Pharmacol. Sci. 15:124-129, 1994; Hruby, Biopolymers 33:1073-1082, 1993; Bugg et al., Sci. Am. 269:92-98, 1993, all incorporated herein by reference).

Thus, through use of the methods described above, the present invention provides compounds exhibiting enhanced therapeutic activity in comparison to the polypeptides described above. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isosteric pseudopeptide bond is a suitable pseudopeptide bond that is known to enhance stability to enzymatic cleavage with no or little loss of biological activity (Couder et al., Int. J. Polypeptide Protein Res. 41:181-184, 1993, incorporated herein by reference). Thus, the amino acid sequences of these compounds can be identical to the sequences of their parent L-amino acid polypeptides, except that one or more of the peptide bonds are replaced by an isosteric pseudopeptide bond. Preferably the most N-terminal peptide bond is substituted, since such a substitution would confer resistance to proteolysis by exopeptidases acting on the N-terminus

To confer resistance to proteolysis, peptide bonds can also be substituted by retro-inverso pseudopeptide bonds (Dalpozzo et al., Int. J. Polypeptide Protein Res. 41:561-566, incorporated herein by reference). According to this modification, the amino acid sequences of the compounds can be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid derivatives of polypeptides represent another form of modified polypeptides that retain the important structural determinants for biological activity, yet eliminate the peptide bonds, thereby conferring resistance to proteolysis (Simon et al., Proc. Natl. Acad. Sci. USA 89:9367-9371, 1992, and incorporated herein by reference). Peptoids are oligomers of N-substituted glycines. A number of N-alkyl groups have been described, each corresponding to the side chain of a natural amino acid.

Polynucleotides

The invention includes polynucleotides encoding peptides of the invention. Exemplary polynucleotides encode peptides including those described herein, and analogs, derivatives, amidated variations and conservative variations thereof, wherein the peptides have biological activity as described herein. The peptides of the invention include those described herein, as well as the broader groups of peptides having hydrophilic and hydrophobic substitutions, and conservative variations thereof.

“Isolated” when used in reference to a polynucleotide, refers to a polynucleotide substantially free of proteins, lipids, nucleic acids, for example, with which it is naturally associated. As used herein, “polynucleotide” refers to a polymer of deoxyribonucleotides or ribonucleotides, in the form of a separate fragment or as a component of a larger construct. DNA encoding a peptide of the invention can be assembled from cDNA fragments or from oligonucleotides which provide a synthetic gene which is capable of being expressed in a recombinant transcriptional unit. Polynucleotide sequences of the invention include DNA, RNA and cDNA sequences. A polynucleotide sequence can be deduced from the genetic code, however, the degeneracy of the code must be taken into account. Polynucleotides of the invention include sequences which are degenerate as a result of the genetic code. Such polynucleotides are useful for the recombinant production of large quantities of a peptide of interest, such as the peptides described herein.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

In the present invention, the polynucleotides encoding the peptides of the invention can be inserted into a recombinant “expression vector”. The term “expression vector” refers to a plasmid, virus or other vehicle known in the art that has been manipulated by insertion or incorporation of genetic sequences. Such expression vectors of the invention are preferably plasmids that contain a promoter sequence that facilitates the efficient transcription of the inserted genetic sequence in the host. The expression vector typically contains an origin of replication, a promoter, as well as specific genes that allow phenotypic selection of the transformed cells. For example, the expression of the peptides of the invention can be placed under control of E. coli chromosomal DNA comprising a lactose or lac operon which mediates lactose utilization by elaborating the enzyme beta-galactosidase. The lac control system can be induced by IPTG. A plasmid can be constructed to contain the lac Iq repressor gene, permitting repression of the lac promoter until IPTG is added. Other promoter systems known in the art include beta lactamase, lambda promoters, the protein A promoter, and the tryptophan promoter systems. While these are the most commonly used, other microbial promoters, both inducible and constitutive, can be utilized as well. The vector contains a replicon site and control sequences which are derived from species compatible with the host cell. In addition, the vector can carry specific gene(s) which are capable of providing phenotypic selection in transformed cells. For example, the beta-lactamase gene confers ampicillin resistance to those transformed cells containing the vector with the beta-lactamase gene. An exemplary expression system for production of the peptides of the invention is described in U.S. Pat. No. 5,707,855.

Transformation of a host cell with the polynucleotide can be carried out by conventional techniques known to those skilled in the art. For example, where the host is prokaryotic, such as E. coli, competent cells that are capable of DNA uptake can be prepared from cells harvested after exponential growth and subsequently treated by the CaCl₂ method using procedures known in the art. Alternatively, MgCl₂ or RbCl could be used.

In addition to conventional chemical methods of transformation, the plasmid vectors of the invention can be introduced into a host cell by physical means, such as by electroporation or microinjection. Electroporation allows transfer of the vector by high voltage electric impulse, which creates pores in the plasma membrane of the host and is performed according to methods known in the art. Additionally, cloned DNA can be introduced into host cells by protoplast fusion, using methods known in the art.

DNA sequences encoding the peptides can be expressed in vivo by DNA transfer into a suitable host cell. “Host cells” of the invention are those in which a vector can be propagated and its DNA expressed. The term also includes any progeny of the subject host cell. It is understood that not all progeny are identical to the parental cell, since there can be mutations that occur during replication. However, such progeny are included when the terms above are used. Preferred host cells of the invention include E. coli, S. aureus and P. aeruginosa, although other Gram-negative and Gram-positive organisms known in the art can be utilized as long as the expression vectors contain an origin of replication to permit expression in the host.

The polynucleotide sequence encoding the peptide used according to the method of the invention can be isolated from an organism or synthesized in the laboratory. Specific DNA sequences encoding the peptide of interest can be obtained by:1) isolation of a double-stranded DNA sequence from the genomic DNA; 2) chemical manufacture of a DNA sequence to provide the necessary codons for the peptide of interest; and 3) in vitro synthesis of a double-stranded DNA sequence by reverse transcription of mRNA isolated from a donor cell. In the latter case, a double-stranded DNA complement of mRNA is eventually formed that is generally referred to as cDNA.

The synthesis of DNA sequences is frequently the method of choice when the entire sequence of amino acid residues of the desired peptide product is known. In the present invention, the synthesis of a DNA sequence has the advantage of allowing the incorporation of codons that are more likely to be recognized by a bacterial host, thereby permitting high level expression without difficulties in translation. In addition, virtually any peptide can be synthesized, including those encoding natural peptides, variants of the same, or synthetic peptides.

Pharmaceutical Compositions

Methods for treatment of diseases and disorders with pharmaceutical compositions of the present invention are also encompassed by the present invention. Said methods of the invention can include administering a therapeutically effective amount of cells or CM of the present invention. The cells or CM of the invention can be formulated in pharmaceutical compositions. These compositions can comprise, in addition to one or more of the cells or CM, a pharmaceutically acceptable excipient, carrier, buffer, stabiliser or other materials well known to those skilled in the art. Such materials should typically be non-toxic and should not interfere with the efficacy of the active ingredient. The precise nature of the carrier or other material can depend on the route of administration, e.g., oral, intravenous, cutaneous or subcutaneous, nasal, intramuscular, or intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

Whether it is a cell, CM, or other pharmaceutically useful compound according to the present invention that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, current edition (Easton, Pa.:Mack Publishing Company).

A composition of the present invention can be administered alone or in combination with other treatments, either simultaneously or sequentially dependent upon the condition to be treated.

Exemplary Methods

Administration

In certain aspects, an effective amount of BCL2 (for example, BCL2A1) contacted cells or CM is delivered to a subject in need thereof for treating or preventing a disease or disorder. In other aspects, a therapeutically effective amount of CM or cells are administered to the subject. Therapeutically effective amounts of cells and CM in any combination thereof can also be administered. An effective amount for treatment can be determined, e.g., by the body weight of the subject receiving treatment, and can be further modified, for example, based on the severity of the condition, the phase of condition in which therapy is initiated, for example early or advanced, and the simultaneous presence or absence of multiple conditions. The therapeutic amount can also be determined based on the method of delivery to the subject. The therapeutic amount can be one or more administrations of the therapy. Administration of the therapeutic amount of cells or CM can be via continuous infusion, for example, but not limited to a period of 24 hours. In one aspect, about 0.1 to about 2.0 ml/kg body weight CM can be administered in a therapeutic dose, or about 0.4 to about 1.0 ml/kg body weight CM can be delivered in a therapeutic dose, although other does are possible as deemed reasonable to a medical practitioner. In one aspect, about 0.01 to about 5×10⁶ cells per kilogram of recipient body weight can be administered in a therapeutic dose, or about 0.02 to about 1×10⁶ cells per kilogram of recipient body weight can be administered in a therapeutic dose. The number of cells used can depend on the weight and condition of the recipient, the number of or frequency of administrations, and other variables known to those of skill in the art. For example, a dose can be 1, 2, 3, 4, 5, 6, 7, 8, 9, or more administrations of the composition. A subsequent dose can include a dose of cells or CM, or combinations thereof. The therapeutic amount of contacted cells or CM can be administered to the subject prior to an event inducing the need for treatment, for example, prior to surgery, treatment with chemotherapy, heart surgery, heart therapy, and the like.

In one aspect, cells and/or CM can be administered to the patient by injection or instillation intravenously (i.e., large central vein such vena cava) or intra-arterially (i.e., via femoral artery into supra-renal aorta). Any delivery method, commonly known in the art, can be used for delivery of the cells or CM. Administration routes can include:mucosal, parenteral, transdermal, subcutaneous, intravenous, bolus injection, intramuscular, or intraarterial administration to the subject.

Since cells can be expanded in vitro and CM can be collected and stored, multiple administrations of cells and/or CM are possible to further augment the therapeutic effect of the cells and CM. Subject populations that can benefit from administration of cells and CM include, but are not limited to:trauma or surgical patients, e.g., scheduled to undergo high risk surgery such as the repair of an aortic aneurysm, and patients having reperfusion injury. As discussed above, administration of the therapeutic amount of cells or CM can be prior to, during or post development of the condition requiring treatment. Multiple therapeutic amounts can be given to the subjects. Cells and CM therapies can be used for conditions involving any injured organs, including the kidney, lungs, liver, heart, blood, arteries, veins, and the like

Assessment of the outcome of the administration of the therapeutically effective amount of cells or CM can be assessed by techniques commonly known to one of skill in the art. For example, biopsy samples of tissues can be measured and analyzed in the patients and experimental models before and after treatment.

Diseases and Disorders

Diseases and disorders treatable or preventable with the present invention are numerous. Examples of diseases and disorders can include:ischemia and reperfusion injury, ischemia, reperfusion injury, myocardial infarction, graft rejection, skin-graft rejection, cancer, melanoma, acute renal failure, multiple sclerosis, diabetes, rheumatoid arthritis (RA), retinal degeneration, acute lung injury, radiation exposure/trauma, insulin-dependent (Type 1) diabetes and/or hepatic failure.

Inflammation is known to occur in many diseases and disorders treatable or preventable with the present invention, which include, but are not limited to the following:Systemic Inflammatory Response (SIRS); Alzheimer's Disease (and associated conditions and symptoms including:chronic neuroinflammation, glial activation; increased microglia; neuritic plaque formation; and response to therapy); Amyotropic Lateral Sclerosis (ALS), arthritis (and associated conditions and symptoms including, but not limited to:acute joint inflammation, antigen-induced arthritis, arthritis associated with chronic lymphocytic thyroiditis, collagen-induced arthritis, juvenile arthritis; rheumatoid arthritis, osteoarthritis, prognosis and streptococcus-induced arthritis, spondyloarthopathies, gouty arthritis), asthma (and associated conditions and symptoms, including:bronchial asthma; chronic obstructive airway disease; chronic obstructive pulmonary disease, juvenile asthma and occupational asthma); acute and chronic lung disease, cardiovascular diseases (and associated conditions and symptoms, including atherosclerosis; autoimmune myocarditis, chronic cardiac hypoxia, acute and chronic heart failure, congestive heart failure, coronary artery disease, cardiomyopathy and cardiac cell dysfunction, including:aortic smooth muscle cell activation; cardiac cell apoptosis; and immunomodulation of cardiac cell function; diabetes and associated conditions and symptoms, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, acute and chronic liver disease, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1 induced plasticity of hypothalamic neurons and chronic stress hyperresponsiveness), ophtlialmopathies (and associated diseases and conditions, including diabetic retinopathy, Graves' opthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media (adult or pediatric), pancreatitis or pancreatic acinitis, periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis Immunological disorders, including autoimmune diseases, such as alopecia aerata, autoimmune myocarditis, Graves' disease, Graves opthalmopathy, lichen sclerosis, multiple sclerosis, psoriasis, systemic lupus erythematosus, systemic sclerosis, thyroid diseases (e.g., goiter and struma lymphomatosa (Hashimoto's thyroiditis, lymphadenoid goiter), sleep disorders and chronic fatigue syndrome and obesity (non-diabetic or associated with diabetes). Resistance to infectious diseases, such as Leishmaniasis, Leprosy, Lyme Disease, Lyme Carditis, malaria, cerebral malaria, meningitis, tubulointerstitial nephritis associated with malaria), which are caused by bacteria, viruses (e.g., cytomegalovirus, encephalitis, Epstein-Barr Virus, Human Immunodeficiency Virus, Influenza Virus) or protozoans (e.g., Plasmodium falciparum, trypanosomes). Response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g., cerebral palsy), lung injury (acute hemorrhagic lung injury, Goodpasture's syndrome, acute ischemic reperfusion), myocardial dysfunction, caused by occupational and environmental pollutants (e.g., susceptibility to toxic oil syndrome silicosis), radiation trauma, efficiency of responses (e.g., burn or thermal wounds, chronic wounds, surgical wounds, spinal cord injuries, spinal fluid in trauma), and vitreal injection in AMD. Hormonal regulation including fertility/fecundity, likelihood of a pregnancy, incidence of preterm labor, prenatal and neonatal complications including preterm low birth weight, cerebral palsy, septicemia, hypothyroidism, oxygen dependence, cranial abnormality, early onset menopause. A subject's response to transplant (rejection or acceptance), acute phase response (e.g., febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response. These and other diseases and disorders can be treatable or are preventable.

Other diseases or disorders treatable or preventable through use of compositions of the present invention are generally discernable to one of skill in the art without undue experimentation.

The pharmaceutical compositions are generally formulated as sterile, substantially isotonic and in full compliance with all Good Manufacturing Practice (GMP) regulations of the U.S. Food and Drug Administration.

Kits

For use in research and therapeutic applications suggested above, kits are also provided by the invention. In the diagnostic and research applications such kits can include any or all of the following: assay reagents, buffers BCL proteins, hybridization probes and/or primers, BCL variant polypeptides or polynucleotides, and the like. A therapeutic product can include sterile saline or another pharmaceutically acceptable emulsion and suspension base as described above.

Accordingly, kits of the present invention can contain any reagent used to treat diseases and disorders as described herein. Kits of the present invention can also contain additional agents that can be administered concomitantly with the compositions of the present invention.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

From the foregoing description, various modifications and changes in the compositions and methods will occur to those skilled in the art. All such modifications coming within the scope of the appended claims are intended to be included therein. Each recited range includes all combinations and sub-combinations of ranges, as well as specific numerals contained therein.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

### EXEMPLARY ASPECTS

Below are examples of specific aspects for carrying out the present invention. The examples are offered for illustrative purposes only, and are not intended to limit the scope of the present invention in any way. Efforts have been made to ensure accuracy with respect to numbers used (e.g., amounts, temperatures, and the like), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins:Structures and Molecular Properties (W.H. Freeman and Company, current edition); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, current edition (Easton, Pa.:Mack Publishing Company); Carey and Sundberg Advanced Organic Chemistry, current edition (Plenum Press).

Methods

Cell and CM (Supernatant) Preparation

Myeloid cells, 6.0×10⁷, were incubated in 18 ml of complete medium containing 300 ng/ml of rhBCL2A1 (or saline) and incubated at 37° C. for 4 hours. Cells were then washed 3 times and injected intra-peritoneal (i.p.) into mice. The following day the mice were subjected to ischemia-reperfusion injury as described below. In separate experiments, the supernatant from myeloid cells (JawsII or THP-1 or bone marrow derived macrophages) previously incubated with rhBCL2A1 was given to mice that were then subjected to ischemia-reperfusion. Cell preparation and ischemia-reperfusion injury is described below.

MSCs, 6×10⁶, Jaws-II and THP-1 cells, 6.0×10⁷, were incubated in 18 ml of complete medium containing 300 ng/ml of rhBCL2A1 or 300 ng/ml rhBim or saline at 37° C. for 4 hours then the cells were washed 3 times then were cultured in medium without serum an additional 18 hours at 37° C. The medium was collected by centrifugation and concentrated (approximately 1:100) by ultra-filtration using a 5000 Dalton cut-off filter. The supernatant was adjusted to 6 ml and 1 ml given to each mouse and on the following day they were subjected to ischemia-reperfusion injury.

Bone marrow derived macrophages were produced by harvesting the marrow from 2 C57B1/6 mice and divided between 12 petri dishes containing 7 ml of medium containing fetal bovine serum, L-glutamine and 50 μg/ml rhM-CSF per dish and incubated for 3 days. On the 4^{th} day each dish was washed twice with RPMI and again cultured in the presence of rhM-CSF for an additional 2 days. On the 6^{th} day, cells were removed from the dish counted and 6.0×10⁷, were incubated in 18 ml of medium containing rhM-CSF. The next day rhBim or rhBCL2A1 (300 ng/ml final concentration) was added to the cells. After 4 hours of additional incubation, the cells were washed 3 times and incubated overnight in medium without serum or rhM-CSF. Following this incubation, the medium was collected by centrifugation and concentrated (approximately 1:100) by ultra-filtration using a 5000 Dalton cut-off filter. The supernatant was adjusted to 6 ml and 1 ml given to each mouse and on the following day they were subjected to ischemia-reperfusion injury.

Hind Limb Ischemia-Reperfusion

Ischemia-reperfusion was accomplished by applying a tourniquet to both hind limbs of mice. Bilateral tourniquets (Latex O-rings) were applied above the greater trochanter using the McGivney Hemorrhoid Ligator (Miltex, York, Pa.). Mice were maintained in a supine position under halothane or isoflurane in an incubator at 30° C. during the 90-minute ischemic period.

The hind limb tourniquet was removed at the end of ischemia to establish reperfusion and mice were allowed to recover in the 30° C. incubator in order to maintain normal body temperature. Reperfusion was continued for 24 hours and injury was assessed by mitochondrial function. At the end of reperfusion, mice were killed, the previously ischemic muscle removed, and tissue viability measured using 3-(4,5-dimethylthiazol-2-yl)-2,5-diphenyl tetrazolium (MTT). Briefly, the excised muscle tissue was cut into pieces to increase the surface area and uptake of MTT. The tissue sections were placed in 3 ml of PBS supplemented with 60 μl of 5 mg/ml MTT and incubated for 3 hours at 37° C. on a shaker. Samples were removed, blotted dry and the formazam salt extracted in 3 ml of 2-propanol overnight at 37° C. in the dark. Absorbance at 570 nm of 200 nl of the solution was determined and normalized to the dry weight of the tissue. Viable mitochondria reduce MTT to a water-insoluble salt that is soluble in isopropanol and can be extracted by isopropanol. Live tissue with functional mitochondria therefore has an increased optical density relative to dead tissue.

### Example 1

### Adoptive Transfer of rhBCL2A1 Treated Myeloid Cells Provides Protection From Ischemia-Reperfusion Injury

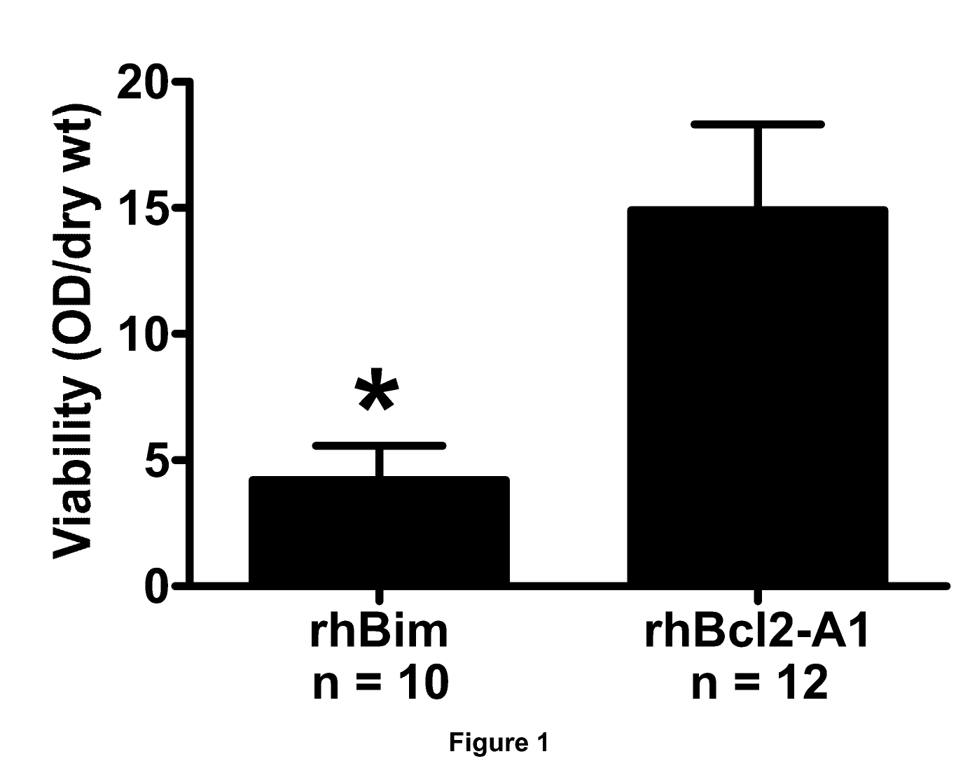

This Example was performed using the methods described above. FIG. 1 shows mouse hind limb tissue viability as measured by MTT assay following treatment of the mice with Jaws-II cells (available from ATCC) previously incubated with either recombinant human (rh)BCL2A1 or rhBim. The cells treated with rhBCL2A1 provided protection from 90 minutes of ischemia followed by 24 hours of reperfusion. Differences were significant by Mann-Whitney test at p<0.05.

### Example 2

### Supernatant From rhBcl-2-A1 treated Jaws-II Cells Provides Protection From Ischemia-Reperfusion Injury

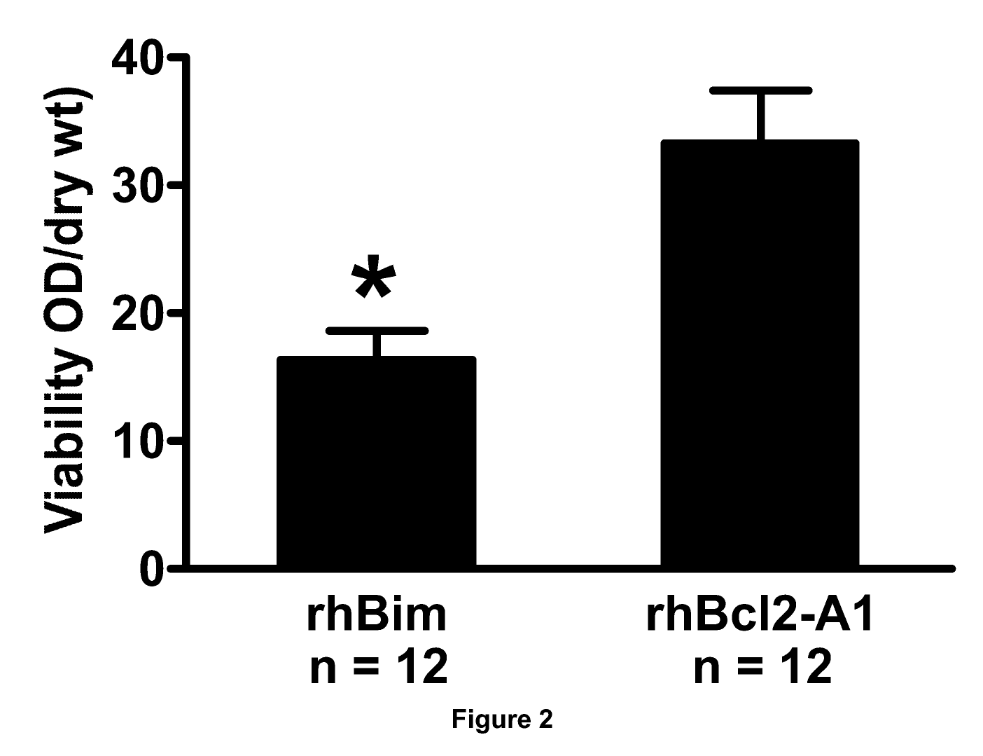

This Example was performed using the methods described above. FIG. 2 shows mouse hind limb tissue viability as measured by MTT assay following treatment with supernatant from Jaws-II cells previously incubated with either rhBCL2A1 or rhBim. Supernatant from rhBCL2A1 treated Jaws-II cells provided protection from 90 minutes of ischemia followed by 24 hours of reperfusion. Differences were significant by t-test at p<0.05.

### Example 3

### Supernatant From rhBcl-2-A1 treated Human THP-1 Cells Provides Protection From Ischemia-Reperfusion Injury

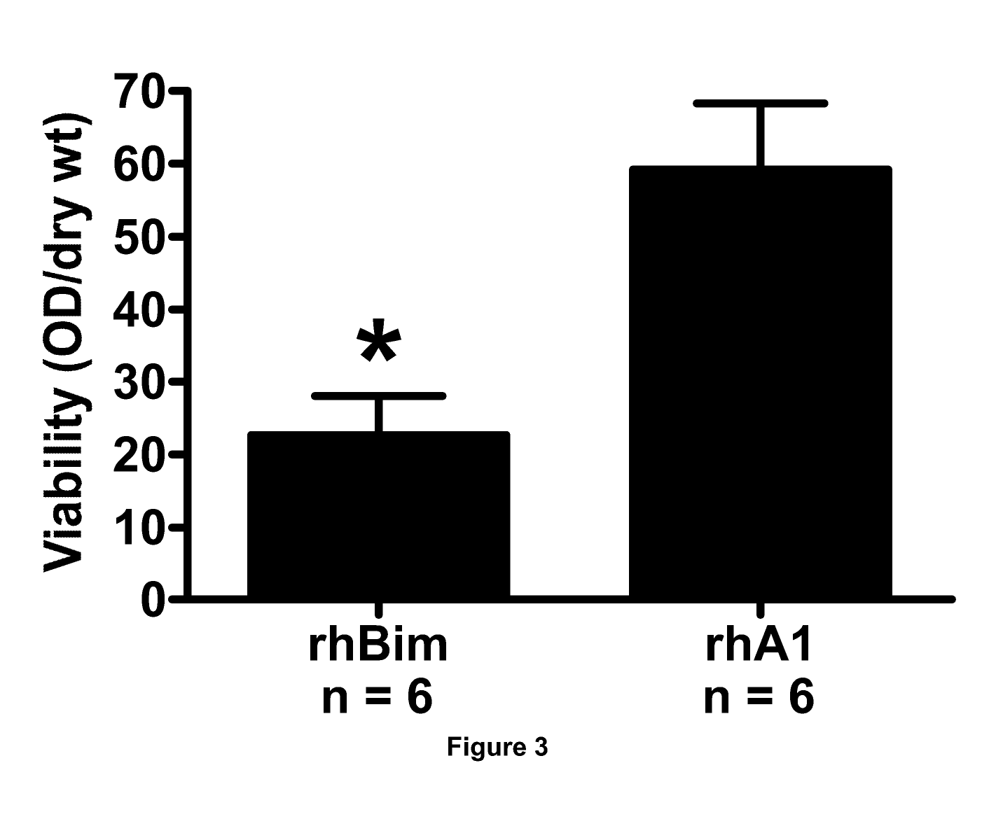

This Example was performed using the methods described above. FIG. 3 shows mouse hind limb tissue viability as measured by MTT assay following treatment with supernatant from human THP-1 cells previously incubated with either rhBCL2A1 or rhBim. Supernatant from rhBCL2A1 treated THP-1 cells provided protection from 90 minutes of ischemia followed by 24 hours of reperfusion. Differences were significant by t-test at p<0.05.

### Example 4

### Supernatant From rhBcl-2-A1 treated BMDM Provides Protection from Ischemia-Reperfusion Injury

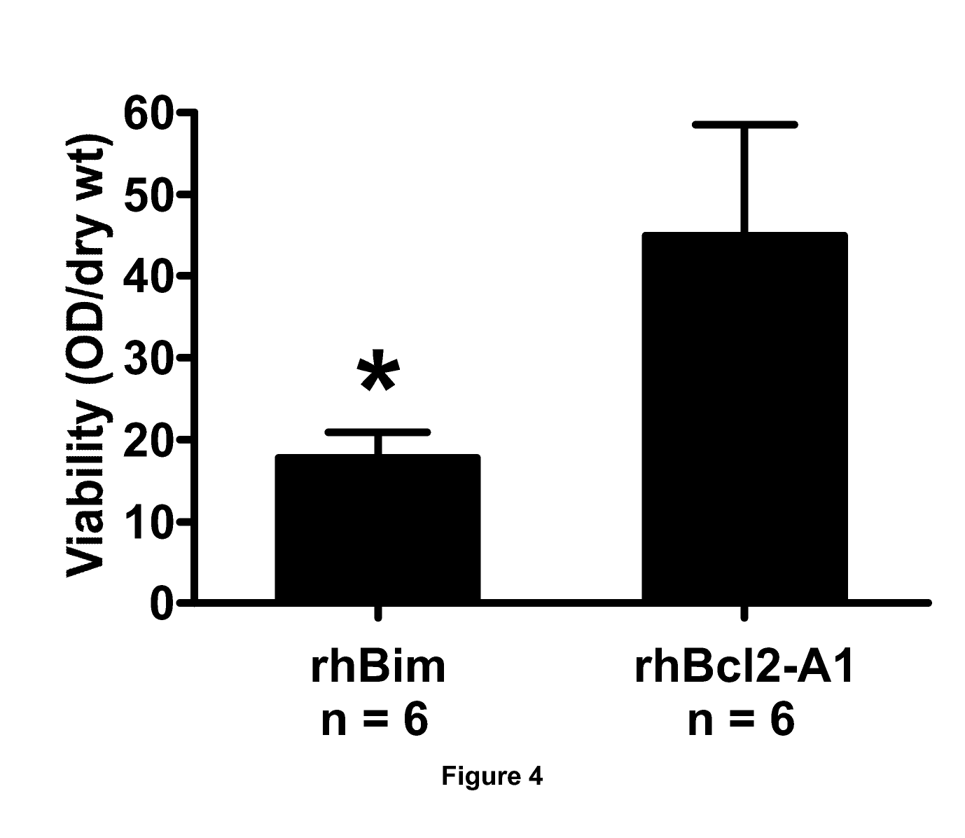

This Example was performed using the methods described above. FIG. 4 shows mouse hind limb tissue viability as measured by MTT assay following treatment with supernatant from bone marrow derived macrophages (BMDMs) previously incubated with either rhBCL2A1 or rhBim. Supernatant from rhBCL2A1 treated BMDM provided protection from 90 minutes of ischemia followed by 24 hours of reperfusion. Differences were significant by Mann-Whitney test at p<0.05.

### Example 5

### Supernatant From rhBcl-2-A1 treated MSCs Provides Protection from Ischemia-Reperfusion Injury

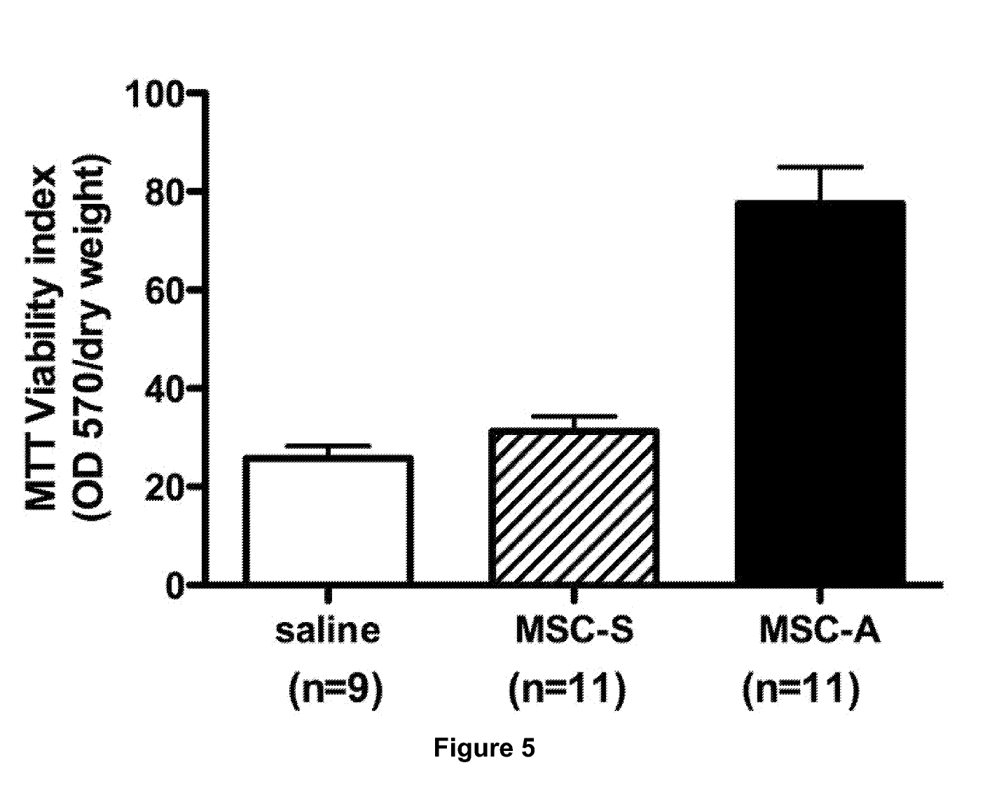

This Example was performed using the methods described above. FIGS. 5 and 6 show mouse hind limb tissue viability as measured by MTT assay following treatment with supernatant from mesenchymal stem cells (MSCs) previously incubated with either rhBCL2A1 or saline. Supernatant from rhBCL2A1 treated MSCs provided protection from 90 minutes of ischemia followed by 24 hours of reperfusion. Differences were significant by ANOVA and the Non-parametric test at p<0.0001.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

INFORMAL SEQUENCE LISTING

| SEQ ID NO | DESCRIPTION | SEQUENCE |
|---|---|---|
| SEQ ID NO: 14 | BCL2A1 nucleotide sequence; | agcctacgca cgaaagtgac taggaggaag |
| | Genbank no. NM_004049 | gatattataa agtgatgcaa acagaaattc |
| | | caccagcctc catgtatcat catgtgtcat |
| | | aactcagtca agctcagtga gcattctcag |
| | | cacattgcct caacagcttc aaggtgagcc |
| | | agctcaagac tttgctctcc accaggcaga |
| | | agatgacaga ctgtgaattt ggatatattt |
| | | acaggctggc tcaggactat ctgcagtgcg |
| | | tcctacagat accacaacct ggatcaggtc |
| | | caagcaaaac gtccagagtg ctacaaaatg |
| | | ttgcgttctc agtccaaaaa gaagtggaaa |
| | | agaatctgaa gtcatgcttg gacaatgtta |
| | | atgttgtgtc cgtagacact gccagaacac |
| | | tattcaacca agtgatggaa aaggagtttg |
| | | aagacggcat cattaactgg ggaagaattg |
| | | taaccatatt tgcatttgaa ggtattctca |
| | | tcaagaaact tctacgacag caaattgccc |
| | | cggatgtgga tacctataag gagatttcat |
| | | attttgttgc ggagttcata atgaataaca |
| | | caggagaatg gataaggcaa aacggaggct |
| | | gggaaaatgg ctttgtaaag aagtttgaac |
| | | ctaaatctgg ctggatgact tttctagaag |
| | | ttacaggaaa gatctgtgaa atgctatctc |
| | | tcctgaagca atactgttga ccagaaagga |
| | | cactccatat tgtgaaaccg gcctaatttt |
| | | tctgactgat atggaaacga ttgccaacac |
| | | atacttctac ttttaaataa acaactttga |
| | | tgatgtaact tgaccttcca gagttatgga |
| | | aattttgtcc ccatgtaatg aataaattgt |
| | | atgtattttt ctctataaaa aaaaaaaaa |
| SEQ ID NO: 15 | BCL2A1 protein sequence | MTDCEFGYIYRLAQDYLQCVLQIPQPGSGPSK |
| | | TSRVLQNVAFSVQKEVEKNLKSCLDNVNVVSV |
| | | DTARTLFNQVMEKEFEDGIINWGRIVTIFAFE |
| | | GILIKKLLRQQIAPDVDTYKEISYFVAEFIMN |
| | | NTGEWIRQNGGWENGFVKKFEPKSGWMTFLEV |
| | | TGKICEMLSLLKQYC |

What is claimed is:

1. A method of treating or preventing a disease or disorder, comprising: administering a therapeutically or prophylactically effective amount of a composition to a subject comprising the disease or disorder, wherein the composition comprises an isolated cell contacted with BCL2 or a supernatant component, wherein the supernatant component is derived from a cell culture contacted with BCL2.

2. The method of claim 1, wherein BCL2 is BCL2A1.

3. The method of claim 1, wherein BCL2 is a protein comprising at least 12 amino acids, wherein the protein is at least 50% similar to a segment of an A-1 protein, wherein the A-1 protein consists of the amino acid sequence set forth in SEQ ID NO:4.

4. The method of claim 1, wherein the disease or disorder comprises an ischemia and reperfusion injury.

5. The method of claim 1, wherein the disease or disorder comprises ischemia, reperfusion injury, myocardial infarction, graft rejection, skin-graft rejection, cancer, melanoma, acute renal failure, multiple sclerosis, diabetes, rheumatoid arthritis (RA), retinal degeneration, acute lung injury, radiation trauma, insulin-dependent (Type 1) diabetes or hepatic failure.

6. The method of claim 2, wherein BCL2A1 is a recombinant BCL2A1.

7. The method of claim 2, wherein BCL2A1 is a human BCL2A1.

8. The method of claim 1, wherein the cell or cell culture comprises a mesenchymal stem cell (MSC).

9. The method of claim 1, wherein the cell or cell culture comprises a MSC, a dendritic cell, a monocyte, a macrophage, a myeloid cell, a THP-1 cell, a JAWS-II cell, a leukocyte, a stem cell, or an immune cell.

10. The method of claim 1, wherein BCL2 comprises a BCL2A1 protein.

11. The method of claim 1, wherein BCL2 comprises a BCL2A1 nucleic acid.

12. The method of claim 1, wherein the administration comprises mucosal, parenteral, transdermal, subcutaneous, intravenous, bolus injection, intramuscular, or intraarterial administration to the subject.

13. A composition for treating or preventing a disease or disorder, comprising an isolated cell contacted with BCL2.

14. The composition of claim 13, wherein BCL2 is BCL2A1.

15. The composition of claim 13, wherein BCL2 is selected from the group consisting of: a protein comprising at least 12 amino acids, wherein the protein is at least 50% similar to a segment of an A-1 protein, wherein the A-1 protein consists of the amino acid sequence set forth in SEQ ID NO:4.

16. The composition of claim 13, wherein the disease or disorder comprises an ischemia and reperfusion injury.

17. The composition of claim 13, wherein BCL2A1 is a recombinant BCL2A1.

18. The composition of claim 14, wherein the cell comprises a MSC.

19. A composition for treating or preventing a disease or disorder, comprising a supernatant component, wherein the supernatant component is derived from a cell culture contacted with BCL2.

20. The composition of claim 19, wherein the cell culture comprises a MSC.
